(19) United States
(12) Patent Application Publication
(10) Pub. No.: US 2021/0198684 A1
(43) Pub. Date: Jul. 1, 2021

(54) METHOD FOR INCREASING THE YIELD OF MICROALGAE AND PRODUCTS PRODUCED THEREBY, AN ALTERED CAM1 GENE AND POLYPEPTIDE, AND A NOVEL CHLAMYDOMONAS SP.

(71) Applicant: Industrial Technology Research Institute, Hsinchu (TW)

(72) Inventors: Chang-Chieh CHEN, Taipei City (TW); Shao-I WU, Jiaoxi Township (TW); Jo-Di CHIANG, Taipei City (TW); Yu-Pei LIN, Taichung City (TW); Wen-Chang LU, Hsinchu City (TW)

(73) Assignee: Industrial Technology Research Institute, Hsinchu (TW)

(21) Appl. No.: 17/135,402

(22) Filed: Dec. 28, 2020

(30) Foreign Application Priority Data
Dec. 31, 2019 (TW) 108148724

(51) Int. Cl. C12N 15/82; C12N 1/12; C12N 15/113

(52) U.S. Cl. CPC C12N 15/8261; C12N 15/8216; C12N 2310/20; C12N 15/113; C12N 1/12

(57) ABSTRACT

A method for increasing the yield of microalgae and the yield of a product produced by the microalgae is provided. The method includes performing a change procedure on CAM1 gene and/or calmodulin 1 encoded by the CAM1 gene in a microalga, such that a change occurs in a nucleotide and/or the nucleotide sequence of the CAM1 gene and/or an amino acid and/or the amino acid sequence of the calmodulin 1 encoded by the CAM1 gene in the microalga to obtain an altered microalga. The altered microalga has an altered CAM1 gene and/or an altered calmodulin 1. The altered microalga has a higher growth rate and a higher product production rate and/or yield than an unaltered microalga.

### CROSS REFERENCE TO RELATED APPLICATION

The present application is based on, and claims priority from, Taiwan Application Serial Number 108148724, filed on Dec. 31, 2019, the disclosure of which is hereby incorporated by reference herein in its entirety.

### INCORPORATION BY REFERENCE OF SEQUENCE LISTING

A sequence listing submitted as a text file via EFS-Web is incorporated herein by reference. The text file containing the sequence listing is named “9044E-A26958-US_Seq_Listing.txt”; its date of creation is Dec. 23, 2020; and its size is 28,256 bytes.

### TECHNICAL FIELD

The technical field relates to a method for increasing the yield of microalgae and the yield of a product produced by the microalgae, and in particularly relates to a method for increasing the yield of microalgae and the yield of a product produced by the microalgae by changing CAM1 gene in a microalga.

### BACKGROUND

Microalgae can effectively use light energy, carbon dioxide and inorganic salts to synthesize proteins, fats, carbohydrates and a variety of high added value-biologically active substances, and thus microalgae can be used in health foods, food additives, feed and other chemicals production. With the development of bioenergy technology in recent years, some oily microalgae have high oil production capacity (approximately occupies 20-60% of dry cell weight), and have advantages such as high photosynthetic efficiency, short growth cycle, and capable of growing in different geographical environments, and at the same time can absorb carbon dioxide in the exhaust of the factory to achieve the effect of reducing carbon, and thus it is currently the focus of research and development in countries around the world.

However, obtainment of microalgal biomass in large-scale, low-cost, high-efficiency, is currently the biggest bottleneck of microalgal bio-energy industrialization. Based on the analysis, the cost of obtaining microalgal biomass accounts for about 60% of the overall biodiesel production cost. Accordingly, it is the key technology for commercialization of microalgal technology to increase the yield of microalgal cells and related products and shorten the production time.

Therefore, there is an urgent need for a novel method for increasing the yield of microalgae and the yield of a product produced by the microalgae.

### SUMMARY

The present disclosure provides a method for increasing the yield of microalgae and the yield of a product produced by the microalgae, comprising: performing a change procedure on CAM1 gene and/or calmodulin 1 encoded by the CAM1 gene in a microalga, such that a change occurs in a nucleotide and/or the nucleotide sequence of the CAM1 gene and/or an amino acid and/or the amino acid sequence of the calmodulin 1 encoded by the CAM1 gene in the microalga to obtain an altered microalga. The altered microalga has an altered CAM1 gene and/or an altered calmodulin 1. The altered microalgahas a higher growth rate and a higher product production rate and/or yield than an unaltered microalga.

The present disclosure also provides an altered CAM1 gene, of which the nucleotide sequence comprises the sequence of SEQ ID NO. 1, wherein the nucleotide sequence of the CAM1 gene has a change. The region that has a change in the nucleotide sequence of the CAM1 gene comprises at least one of the regions of the sequence of SEQ ID NO. 1 as follows: (i) a region between position 80 and position 90; (ii) a region between position 160 and position 170; (iii) a region between position 230 and position 240; (iv) a region between position 310 and position 320; (v) a region between position 330 and position 340; and (vi) a region between position 335 and position 345.

The present disclosure further provides a polypeptide, of which the amino acid sequence comprises the sequence of SEQ ID NO. 6 or the sequence of SEQ ID NO. 7.

The present disclosure further provides a microalga, which comprises the altered CAM1 gene mentioned above and/or a polypeptide mentioned above.

The present disclosure further provides a novel Chlamydomonas sp., which is Chlamydomonas reinhardtii ITRI-ALG-3 deposited at Bioresource Collection and Research Centre of Food Industry Research and Development Institute and whose deposit number is BCRC 980055 or which is Chlamydomonas reinhardtii ITRI-ALG-8 which is deposited at Bioresource Collection and Research Centre of Food Industry Research and Development Institute and whose deposit number is BCRC 980056.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 shows the expression level of the CAM1 gene of Haematococcus pluvialis stimulated with a microalga growth promoter shown in the transcriptomic analysis;

FIG. 2 shows the effect of changes in the calcium ion concentration of the culture medium on the growth of Haematococcus pluvialis;

FIG. 3 shows the expression level of the CAM1 gene of Chlamydomonas reinhardtii stimulated with a microalga growth promoter shown in the real-time quantitative polymerase chain reaction.

FIG. 4 shows the designs of the guide RNA sequence and the polymerase chain reaction primers on the CAM1 gene fragment of Chlamydomonas sp. used in T7 endonuclease I assay in one embodiment of the present disclosure;

FIG. 5 shows the results of T7 endonuclease I (T7E1) assay for Chlamydomonas reinhardtii transfected with a ribonucleoprotein (RNP) complex in an embodiment of the present disclosure;

FIG. 6 shows the OD₆₈₀ value of the gene-edited candidate strain of Chlamydomonas reinhardiii in a multiwell plate in the high-throughput microalga growth analysis;

FIG. 7 shows the cell numbers of the gene-edited candidate strains 3, 7 and 8 of Chlamydomonas reinhardiii in the growth test of 500 mL serum bottle culture.

FIG. 8 shows the proportions of different-sized populations of cells of the gene-edited candidate strains 3, 7 and 8 of Chlamydomonas reinhardtii in the growth test of 500 mL serum bottle culture;

FIG. 9 shows the chlorophyll content of the gene-edited candidate strains 3, 7 and 8 of Chlamydomonas reinhardiii in the growth test of 500 mL serum bottle culture;

FIG. 10 shows the lipid content and fatty acid methyl ester (FAME) content of the gene-edited candidate strains 3, 7 and 8 of Chlamydomonas reinhardiii in the growth test of 500 mL serum bottle culture;

FIG. 11 shows the sequence alignment result of the sequence of CAM1 gene of the gene-edited candidate strain 3 of Chlamydomonas reinhardtii (SEQ ID NO. 4) and the sequence of CAM1 gene of the wild type strain (SEQ ID NO. 1); and

FIG. 12 shows the sequence alignment result of the sequence of CAM1 gene of the gene-edited candidate strain 8 of Chlamydomonas reinhardtii (SEQ ID NO. 5) and the sequence of CAM1 gene of the wild type strain (SEQ ID NO. 1).

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

In the present disclosure, Haematococcus pluvialis was stimulated with a microalga growth promoter, and then a transcriptomics analysis is performed thereon to screen one of the genes of Haematococcus pluvialis which may be regulated by the microalga growth promoter, CAM1 gene. Next, the transcript database of Haematococcus pluvialis and the genome database of Chlamydomonas sp. were cross-checked, and it is confirmed that a CAM1 gene indeed exists in Chlamydomonas sp. Accordingly, the microalga growth promoter was used to stimulate Chlamydomonas sp. and real-time quantitative polymerase chain reaction (RT-qPCR) was performed to confirm the expression level of the CAM1 gene of Chlamydomonas sp.

The results show that the expression level of CAM1 gene of Chlamydomonas sp. is indeed affected by this microalga growth promoter.

CAM1 gene encodes calmodulin 1, which is a multifunctional intermediate calcium binding messenger protein expressed in eukaryotic cells. In addition, in the present disclosure, since it is confirmed that the difference in calcium ion concentration will indeed affect the growth of microalgae, the CAM1 gene and/or calmodulin 1 encoded thereby is used as a target, and by changing the CAM1 gene and/or calmodulin 1 encoded thereby in a microalga to obtain a modified microalga with improved growth rate and product production rate and/or yield, thereby increasing the yield of microalgae and the yield of the product produced by the microalga.

In view of this, in one embodiment of the present disclosure, a method for increasing the yield of microalgae and the yield of a product produced by the microalgae is provided.

The method for increasing the yield of microalgae and the yield of a product produced by the microalgae of the present disclosure mentioned above may comprise the following steps, but is not limited thereto.

First, a change procedure is performed on CAM1 gene and/or calmodulin 1 encoded by the CAM1 gene in a microalga, such that a change occurs in a nucleotide and/or the nucleotide sequence of the CAM1 gene and/or an amino acid and/or the amino acid sequence of the calmodulin 1 encoded by the CAM1 gene in the microalga to obtain an altered microalga.

The type of the microalga mentioned above is not particularly limited, as long as it has a CAM1 gene, such as Chlamydomonas sp., Haematococcus sp., Synechococcus sp., Volvox sp., Emiliania huxleyi or Heterocapsa triquetra, etc., but it is not limited thereto.

Examples of Chlamydomonas sp. may comprise, but are not limited to, Chlamydomonas reinhardtii, Chlamydomonas acidophila, Chlamydomonas ehrenbergii Gorozhankin, Chlamydomonas moewusii, Chlamydomonas nivalis, Chlamydomonas caudata Wille, Chlamydomonas elegans G. S. West 1915, Chlamydomonas ovoidae, etc., or any combination of thereof.

Examples of Haematococcus sp. may comprise Haematococcus pluvialis, Haematococcus lacustris, Haematococcus zimbabwiensis, Haematococcus capensis, Haematococcus carocellus, Haematococcus droebakensis, Haematococcus murorum, Haematococcus thermalis and the like, or any combination of thereof, but they are not limited thereto.

Examples of Synechococcus sp. may comprise Synechococcus elongatus and the like, but they are not limited thereto.

Examples of Volvox sp. may comprise, but are not limited to, Volvox aureus, Volvox globator, Volvox carteri (Volvox nagariensis), Volvox barberi, Volvox rouseletti, Volvox dissipatrix, Volvox tertius and the like, or any combination thereof.

Moreover, in the foregoing method for increasing the yield of microalgae and the yield of a product produced by the microalgae of the present disclosure, examples of products produced by the microalgae may comprise chlorophyll, a lipid, astaxanthin, a protein, a fatty acid, an amino acid, a carbohydrate, a vitamin, a compound, a cellulose, an enzyme, a colloid, a pigment, diatomaceous earth and the like, or any combination thereof, but they are not limited thereto.

Furthermore, in the foregoing method for increasing the yield of microalgae and the yield of a product produced by the microalgae of the present disclosure, the change procedure mentioned above is not particularly limited, as long as a nucleotide and/or the nucleotide sequence of the CAM1 gene and/or an amino acid and/or the amino acid sequence of the calmodulin 1 encoded by the CAM1 gene in the microalga can be changed, or the CAM1 gene and/or calmodulin 1 can be inactivated. For example, technologies of clustered regularly interspaced short palindromic repeats/CRISPR-associated protein 9 gene editing, zinc-finger nucleases (ZFN) gene editing, or transcription activator-like effector nucleases (TALENs) gene editing and the like can be adopted, but it is not limited thereto. On the other hand, a method, such as traditional mutation, natural mutation, specific or random inactivation, forward or reverse transcription factor labeling, or any method that may affect the normal expression of the protein can be adopted, but it is not limited thereto.

The foregoing change occurring in a nucleotide and/or the nucleotide sequence of the CAM1 gene and/or an amino acid and/or the amino acid sequence of the calmodulin 1 encoded by the CAM1 gene in the microalga is not particularly limited, as long as the growth rate and product production rate and/or yield of microalgae can be increased.

Moreover, the foregoing change occurring in a nucleotide and/or the nucleotide sequence of the CAM1 gene and/or an amino acid and/or the amino acid sequence of the calmodulin 1 encoded by the CAM1 gene in the microalga, may comprise substitution, insertion, deletion, addition, frameshift or any combination thereof in a sequence, and/or comprises modification to a nucleotide or an amino acid, such as methylation of nucleotide, acetylation or hydroxylation of amino acid and the like, but it is not limited thereto.

The foregoing change occurring in a nucleotide and/or the nucleotide sequence of the CAM1 gene and/or an amino acid and/or the amino acid sequence of the calmodulin 1 encoded by the CAM1 gene in the microalga may occur in/at any or a plurality of region or position in a nucleotide and/or the nucleotide sequence of the CAM1 gene and/or an amino acid and/or the amino acid sequence of the calmodulin 1 encoded by the CAM1 gene. Furthermore, the foregoing change may be substitution, insertion, deletion, addition, frameshift and/or any combination thereof, and/or modification that may occur simultaneously in different regions or positions.

In one embodiment, the nucleotide sequence of the CAM1 gene in the microalga mentioned above may comprise the sequence of SEQ ID NO. 1, but it is not limited thereto. The amino acid sequence encoded by the sequence of SEQ ID NO. 1 is the sequence of SEQ ID NO. 2. In one specific embodiment, the nucleotide sequence of the CAM1 gene in the microalga mentioned above may be the sequence of SEQ ID NO. 1.

In the foregoing embodiment, in which the nucleotide sequence of the CAM gene in the microalgae may comprise the sequence of SEQ ID NO. 1, a region or position where the change occurs in the nucleotide sequence of the CAM1 gene may comprise, but is not limited to, at least one of the regions or positions of the sequence of SEQ ID NO. 1 in the following:
(i) a region between position 80 and position 90, such as position 82, but it is not limited thereto;
(ii) a region between position 160 and position 170, such as a region between position 167 and position 168, but it is not limited thereto;
(iii) a region between position 230 and position 240, such as position 233, but it is not limited thereto;
(iv) a region between position 310 and position 320, such as position 313, but it is not limited thereto;
(v) a region between position 330 and position 340, such as position 335, but it is not limited thereto; and
(vi) a region between position 335 and position 345, such as position 340, but it is not limited thereto.

In one embodiment, the nucleotide sequence of the CAM1 gene mentioned above comprises the sequence of SEQ ID NO. 1, and the change occurring in the nucleotide sequence of the CAM1 gene mentioned above may comprise, but is not limited to, at least one change occurring in the sequence of SEQ ID NO. 1 from among the following:
(i) a substitution of a nucleotide at position 82;
(ii) an insertion of at least one nucleotide between position 167 and position 168;
(iii) a substitution of a nucleotide at position 233;
(iv) a substitution of a nucleotide at position 313;
(v) a substitution of a nucleotide at position 335; and
(vi) a substitution of a nucleotide at position 340.

The substitution of a nucleotide at position 82 mentioned above may be a substitution from C to A, but it is not limited thereto.

The insertion of at least one nucleotide between position 167 and position 168 mentioned above may be an insertion of a polynucleotide between position 167 and position 168, but it is not limited thereto. The sequence of the inserted polynucleotide may comprise, but is not limited to, the sequence of SEQ ID NO. 3.

The substitution of a nucleotide at position 233 mentioned above may be a substitution from G to C, but it is not limited thereto.

The substitution of a nucleotide at position 313 mentioned above may be a substitution from T to G, but it is not limited thereto.

The substitution of a nucleotide at position 335 mentioned above may be a substitution from C to G, but it is not limited thereto.

In addition, the substitution of a nucleotide at position 340 mentioned above may be a substitution from A to G, but it is not limited thereto.

In one specific embodiment, the nucleotide sequence of the CAM1 gene mentioned above comprises the sequence of SEQ ID NO. 1, and the change occurring in the nucleotide sequence of the CAM1 gene mentioned above may comprise, but is not limited to, changes occurring in the sequence of SEQ ID NO. 1 in the following:

(i) an insertion of a polynucleotide between position 167 and position 168, in which the sequence of the polynucleotide may be the sequence of SEQ ID NO. 3; and

(ii) a substitution from A to G at position 340.

In another specific embodiment, the nucleotide sequence of the CAM1 gene mentioned above comprises the sequence of SEQ ID NO. 1, and the change occurring in the nucleotide sequence of the CAM1 gene mentioned above may comprise, but is not limited to, changes occurring in the sequence of SEQ ID NO. 1 in the following:

(i) a substitution from C to A at position 82;

(ii) a substitution from G to C at position 233;

(iii) a substitution form T to G at position 313;

(iv) a substitution from C to G at position 335; and

(v) a substitution from A to G at position 340.

In the method for increasing the yield of microalgae and the yield of a product produced by the microalgae of the present disclosure mentioned above, the altered microalga obtained above has an altered CAM1 gene and/or altered calmodulin 1.

The nucleotide sequence of the altered CAM1 gene mentioned above may comprise, but is not limited to, the sequence of SEQ ID NO. 4 or the sequence of SEQ ID NO. 5. The amino acid sequence of the polypeptide encoded by the sequence of SEQ ID NO. 4 is the sequence of SEQ ID NO. 6. Moreover, the amino acid sequence of the polypeptide encoded by the sequence of SEQ ID NO. 5 is the sequence of SEQ ID NO. 7.

The amino acid sequence of the altered calmodulin 1 mentioned above may comprise, but is not limited to, the sequence of SEQ ID NO. 6 or the sequence of SEQ ID NO. 7. The sequence of SEQ ID NO. 6 is encoded by the sequence of SEQ ID NO. 4. Moreover, the sequence of SEQ ID NO. 7 is encoded by the sequence of SEQ ID NO. 5.

In one specific embodiment, in the method for increasing the yield of microalgae and the yield of a product produced by the microalgae of the present disclosure mentioned above, the altered microalga obtained may be Chlamydomonas reinhardtii ITRI-ALG-3, which is deposited at Bioresource Collection and Research Centre (BCRC) of Food Industry Research and Development Institute (FIRDI) and whose deposit number is BCRC 980055.

In another specific embodiment, in the method for increasing the yield of microalgae and the yield of a product produced by the microalgae of the present disclosure mentioned above, the altered microalga obtained may be Chlamydomonas reinhardtii ITRI-ALG-8, which is deposited at Bioresource Collection and Research Centre (BCRC) of Food Industry Research and Development Institute (FIRDI) and whose deposit number is BCRC 980056.

Compared with an unaltered microalga, the foregoing altered microalga has a higher growth rate and a higher product production rate and/or yield, but it is not limited thereto.

According to the foregoing, in another embodiment of the present disclosure, an altered CAM1 gene can be provided.

The nucleotide sequence of the altered CAM1 gene of the present disclosure mentioned above may comprise, but is not limited to, the sequence of SEQ ID NO. 1, and the nucleotide sequence of the altered CAM1 gene mentioned above has a change.

A region or position having the change in the nucleotide sequence of the altered CAM1 gene mentioned above may comprise, but is not limited to, at least one of the regions or positions of the sequence of SEQ ID NO. 1 in the following:

(i) a region between position 80 and position 90, such as position 82, but it is not limited thereto;

(ii) a region between position 160 and position 170, such as a region between position 167 and position 168, but it is not limited thereto;

(iii) a region between position 230 and position 240, such as position 233, but it is not limited thereto;

(iv) a region between position 310 and position 320, such as position 313, but it is not limited thereto;

(v) a region between position 330 and position 340, such as position 335, but it is not limited thereto; and

(vi) a region between position 335 and position 345, such as position 340, but it is not limited thereto.

The foregoing change in the nucleotide sequence of the altered CAM1 gene mentioned above may comprise, but is not limited to, at least one change of the sequence of SEQ ID NO. 1 from among the following:

(i) a substitution of a nucleotide at position 82;

(ii) an insertion of at least one nucleotide between position 167 and position 168;

(iii) a substitution of a nucleotide at position 233;

(iv) a substitution of a nucleotide at position 313;

(v) a substitution of a nucleotide at position 335; and

(vi) a substitution of a nucleotide at position 340.

The substitution of a nucleotide at position 82 mentioned above may be a substitution from C to A, but it is not limited thereto.

The insertion of at least one nucleotide between position 167 and position 168 mentioned above may be an insertion of a polynucleotide between position 167 and position 168, but it is not limited thereto. The sequence of the inserted polynucleotide may comprise, but is not limited to, the sequence of SEQ ID NO. 3.

The substitution of a nucleotide at position 233 mentioned above may be a substitution from G to C, but it is not limited thereto.

The substitution of a nucleotide at position 313 mentioned above may be a substitution from T to G, but it is not limited thereto.

The substitution of a nucleotide at position 335 mentioned above is a substitution from C to G, but it is not limited thereto.

In addition, the substitution of a nucleotide at position 340 mentioned above is a substitution from A to G, but it is not limited thereto.

In one specific embodiment, the foregoing change in the nucleotide sequence of the altered CAM1 gene mentioned above may comprise, but is not limited to, changes of the sequence of SEQ ID NO. 1 in the following:

(i) an insertion of a polynucleotide between position 167 and position 168, in which the sequence of the polynucleotide may be the sequence of SEQ ID NO. 3; and

(ii) a substitution from A to G at position 340.

In another specific embodiment, the foregoing change in the nucleotide sequence of the altered CAM1 gene mentioned above may comprise, but is not limited to, changes of the sequence of SEQ ID NO. 1 in the following:

(i) a substitution from C to A at position 82;

(ii) a substitution from G to C at position 233;

(iii) a substitution from T to G at position 313;

(iv) a substitution from C to G at position 335; and

(v) a substitution from A to G at position 340.

Furthermore, in one specific embodiment, the nucleotide sequence of the CAM1 gene of the present disclosure mentioned above may comprise the sequence of SEQ ID NO. 4, but it is not limited thereto.

In another specific embodiment, the altered CAM1 gene of the present disclosure mentioned above may encode a polypeptide, and the amino acid sequence of the polypeptide may comprise the sequence of SEQ ID NO. 6, but it is not limited thereto.

In addition, in one specific embodiment, the nucleotide sequence of the CAM1 gene of the present disclosure mentioned above may comprise the sequence of SEQ ID NO. 5, but it is not limited thereto.

In another specific embodiment, the altered CAM1 gene of the present disclosure mentioned above may encode a polypeptide, and the amino acid sequence of the polypeptide may comprise the sequence of SEQ ID NO. 7, but it is not limited thereto.

Furthermore, in another embodiment of the present disclosure, a polypeptide can also be provided, in which the amino acid sequence of the polypeptide may comprise the sequence of SEQ ID NO. 6 or the sequence of SEQ ID NO. 7, but it is not limited thereto.

In yet another embodiment of the present disclosure, a microalga may be provided, which may comprise any altered CAM1 genes provided by the present disclosure and/or polypeptide provided by the present disclosure mentioned above, but it is not limited thereto.

The microalga of the present disclosure mentioned above may comprise Chlamydomonas sp., Haematococcus sp., Synechococcus sp., Volvox sp., Emiliania huxleyi or Heterocapsa triquetra, etc., but it is not limited thereto.

Examples of Chlamydomonas sp. of the present disclosure mentioned above may comprise, but are not limited to, Chlamydomonas reinhardtii, Chlamydomonas acidophila, Chlamydomonas ehrenbergii Gorozhankin, Chlamydomonas moewusii, Chlamydomonas nivalis, Chlamydomonas caudata Wille, Chlamydomonas elegans G. S. West 1915, Chlamydomonas ovoidae, etc., or any combination thereof.

Examples of Haematococcus sp. of the present disclosure mentioned above may comprise Haematococcus pluvialis, Haematococcus lacustris, Haematococcus zimbabwiensis, Haematococcus capensis, Haematococcus carocellus, Haematococcus droebakensis, Haematococcus murorum, Haematococcus thermalis and the like, or any combination thereof, but they are not limited thereto.

Examples of Synechococcus sp. of the present disclosure mentioned above may comprise Synechococcus elongatus and the like, but they are not limited thereto.

Examples of Volvox sp. of the present disclosure mentioned above may comprise, but are not limited to, Volvox aureus, Volvox globator, Volvox carteri (Volvox nagariensis), Volvox barberi, Volvox rouseletti, Volvox dissipatrix, Volvox tertius and the like, or any combination thereof.

Moreover, in one specific embodiment, the foregoing microalga provided by the present disclosure may be Chlamydomonas reinhardtii ITRI-ALG-3, which is deposited at Bioresource Collection and Research Centre (BCRC) of Food Industry Research and Development Institute (FIRDI) on Dec. 27, 2019 and whose deposit number is BCRC 980055.

In another specific embodiment, the foregoing microalga provided by the present disclosure may be Chlamydomonas reinhardtii ITRI-ALG-8, which is deposited at Bioresource Collection and Research Centre of Food Industry Research and Development Institute on Dec. 27, 2019 and whose deposit number is BCRC 980056.

In addition, in yet another embodiment of the present disclosure, the present disclosure may provide a novel Chlamydomonas sp., which is Chlamydomonas reinhardtii ITRI-ALG-3 deposited at Bioresource Collection and Research Centre of Food Industry Research and Development Institute on Dec. 27, 2019 and whose deposit number is BCRC 980055 or which is Chlamydomonas reinhardtii ITRI-ALG-8 deposited at Bioresource Collection and Research Centre of Food Industry Research and Development Institute on Dec. 27, 2019 and whose deposit number is BCRC 980056.

### Examples

A. Materials and Methods

A-1. Culture of Haemarococcus pluvialis

1. Haematococcus pluvialis was cultured to stationary phase according to the culture conditions shown in Table 1 below to obtain a suspension containing microalgae in stationary phase.

2. After the suspension was centrifuged at 5,000 rpm and microalgae were obtained, the microalgae were inoculated in a serum bottle containing 500 mL of BG-11 medium to form anmicroalga suspension with a microalga cell number of 1×10⁵. The formula of BG-11 medium is shown in Table 2 below.

TABLE 1
Culture conditions of Haematococcus pluvialis

| Item | Conditions | |
|---|---|---|
| Culture algae name | Haematococcus pluvialis | |
| Culture medium | BG-11 medium | |
| Culture temperature | 25° | C. |
| Illuminance for Culturing | 20,000 | Lux |
| Culture volume | 500 | mL |

| | |
|---|---|
| Days for Culturing | 10 |

TABLE 2
BG-11 medium formula

| | Final | |
|---|---|---|
| Drug Name | concentration | |
| NaNO₃ | 1.76 | mM |
| K₂HPO₄ | 0.23 | mM |
| MgSO₄•7H₂O | 0.3 | mM |
| CaCl₂•2H₂O | 0.24 | mM |
| Citric Acid•H₂O | 0.031 | mM |
| Ferric Ammonium Citrate | 0.021 | mM |
| Na₂EDTA•2H₂O | 0.0027 | mM |
| Na₂CO₃ | 0.19 | mM |

BG-11 trace metal solution (1,000× dilution, per 200 mL)

| | | |
|---|---|---|
| H₃BO₃ | 2.86 | g |
| MnCl₂•4H₂O | 1.81 | g |
| ZnSO₄•7H₂O | 0.222 | g |
| NaMoO₄•2H₂O | 0.39 | g |
| CuSO₄•5H₂O | 0.079 | g |
| Co(NO₃)₂•6H₂O | 49.4 | mg |

A-2. Pre-culture of Chlamydomonas sp.

1. Chlamydomonas sp. was inoculated in 1 L of TAP medium to form an algae suspension with an OD₆₈₀ value of about 0.5. Per liter of TAP medium contained 8.5 mL of phosphate buffer solution, 50 mL of Beijerinck's solution, 1 mL of Hunter's trace solution, and 10 mL of Tris Acetate solution. The formula of phosphoric acid solution, the formula of Beijerinck's solution, the formula of Hunter's trace solution and the formula of Tris Acetate solution are respectively shown in Table 3, Table 4, Table 5 and Table 6 below.

2. The algae suspension mentioned above was cultured under the following conditions. Culturing temperature: 25° C. constant-temperature incubator; aerating gas: 1% CO₂ (1 vvm mixed air); stirring rate: 150 rpm; illumination: 24 hours per day (intensity 10,000 lux).

3. The algae suspension was cultured for about 4 days and when OD₆₈₀ value of about 3 was reached, the algae suspension was used for experiments.

TABLE 3
Formula of phosphate buffer solution

| Ingredient | Content per liter |
|---|---|
| Na₂HPO₄ | 11.62 g |
| KH₂PO₄ | 7.26 g |

TABLE 4
Formula of Beijerinck's solution

| Ingredient | Content per liter |
|---|---|
| NH₄Cl | 8 g/L |
| CaCl₂•2H₂O | 1 g/L |
| MgSO₄•7H₂O | 2 g/L |

TABLE 5
Formula of Hunter's Trace Solution

| Ingredient | Content per liter | |
|---|---|---|
| Na₂EDTA•2H₂O | 50 | g |
| ZnSO₄•7H₂O | 22 | g |
| H₃BO₃ | 11.4 | g |
| MnCl₂•4H₂O | 5.1 | g |
| FeSO₄•7H₂O | 5 | g |
| CoCl₂•6H₂O | 1.6 | g |
| CuSO₄•5H₂O | 1.16 | g |
| (NH₄)₆Mo₇O₂₄•4H₂O | 1.1 | g |

TABLE 6
Formula of Tris Acetate solution

| Ingredient | Content per liter | |
|---|---|---|
| Tris base | 242 | g |
| Glacial acetic acid | 100 | mL |

A-3. Gene Expression Analysis

1. RNA Extraction

For performing the next generation sequencing analysis, total RNA of the sample needs to be extracted. The total RNA of the total sample was extracted with TRIzol (Invitrogen).

(1) 15 mL of the microalga suspension was collected on the Day 0, Day 3, Day 7 and Day 10 of the culturing, respectively.

(2) The microalga suspension was centrifuged and removed the supernatant to obtain the microalga cells.

(3) The microalga cells were re-suspended with 1 mL of DEPC water to obtain a cell suspension.

(4) The cell suspension was added to a mortar and rapidly frozen with liquid nitrogen, and then 1 mL of TRIzol reagent was added to form a mixture solution.

(5) The mixture solution mentioned above was transferred to a 2.0 mL microcentrifuge tube, and 200 μL of chloroform was added therein. After reacting at room temperature for 5 minutes, the microcentrifuge tube was centrifuged at 12,000 g for 15 minutes at 4° C. After centrifugation, the solution was divided into an upper layer of aqueous phase, a middle layer and a lower layer of organic phase.

(6) The upper layer of aqueous phase contained RNA, and the middle layer contained DNA and protein. Approximately 550 μL of the aqueous phase layer was taken and 0.5 mL of isopropanol was added therein and mixed uniformly to form a mixture. After standing at room temperature for 15 minutes, the mixture was centrifuged at 12,000 g for 10 minutes at 4° C. After centrifugation, a white cotton-like RNA precipitate appeared.

(7) After removing the supernatant, 1 mL of 75% RNase free alcohol was added to the precipitate to form a solution to remove excess impurities, and the solution was centrifuged at 7,500 g for 10 minutes.

(8) After removing the supernatant, the precipitate was dried for 5-10 minutes. After that, an appropriate amount of DEPC water was added to re-dissolve the precipitate for determination of RNA quality.

2. Reverse Transcription for cDNA

By using polyT (18) VN as a primer and using MMLV (Invitrogen, Calif., USA) as the reverse transcriptase, the RNA was reverse-transcribed into cDNA.

(1) 1 μL of 100 mM poly (T), 1 μL of 10 mM dNTP Mix (Takara, Japan), and 0 to 11 μL of RNA were mixed to form a mixture.

(2) The mixture mentioned above was placed at 65° C. for 5 minutes, and then placed on ice for rapid cooling.

(3) The above mixture mentioned above was added to 4 μL 5× First-Strand Buffer (Invitrogen, USA) and 2 μL 0.1 M DTT (Invitrogen, USA), and mixed well.

(4) The mixture mentioned above was placed at 37° C. for 2 minutes, and then 1 μL of 200 U/μL MMLV was added therein and mixed well.

(5) The above mixture mentioned above was placed at 37° C. for 50 minutes, and finally, the reaction was terminated by reacting at 70° C. for 15 minutes. During the process, a PCR machine 2720 Thermal cycler (Applied Biosystems, USA) was used to control the temperature.

3. Gene Analysis of Transcripts

In order to analyze the gene regulation mechanism of a microalgae growth promoter for promoting the growth of microalgae, through transcript analysis of next-generation RNA sequencing, the genes with RNA expression more than twice than that on day 0 were screened, and genes with a p value of less than 0.05 were given priority to study.

4. Real-Time Quantitative Polymerase Chain Reaction (qPCR)

(1) RNA concentration of the extracted RNA was quantified using a Qubit 3.0 Fluorometer (Invitrogen) fluorescence analyzer.

(2) 800 ng/μL of RNA was taken and reverse-transcripted by iScript cDNA Synthesis Kit (BIO-RAD) to form a cDNA template.

(3) The sample for quantitative real-time polymerase chain reaction was prepared according to the formula shown in Table 7 below.

(4) Quantitative real-time polymerase chain reaction was performed on the sample mentioned above and the MyGo PCR analysis system was used to monitor the reaction in real time. The conditions for quantitative real-time polymerase chain reaction are shown in Table 8 below.

TABLE 7
Formula of sample for quantitative real-time polymerase chain reaction

| Formula | Volume | |
|---|---|---|
| ddH₂O | 8.2 | μL |
| KAPA SYBR green qPCR Mastermix | | |
| (KAPABIOSYSTEMS) | 10 | μL |
| Forward primer | 0.4 | μL |
| Reverse primer | 0.4 | μL |
| cDNA template | 1 | μL (8 ng/μL) |

TABLE 8
Conditions for quantitative real-time polymerase chain reaction

| Step | Temperature (° C.) | Time (seconds) | Number of cycles |
|---|---|---|---|
| Hold | 95 | 300 | |
| 3 steps of | 95 | 10 | 40 |
| amplification | 58 | 20 | 40 |
| | 72 | 20 | 40 |
| Melting/data | 60 to 97 | | |
| acquisition | | | |

A-4. The Effect of Calcium Ion Concentration of Culture Medium on Haematococcus Pluvialis

1. Microalgae were cultured to stationary phase based on the culture conditions shown in Table 9 below to obtain a suspension containing the microalgae in stationary phase.

2. After the suspension was centrifuged at 5,000 rpm and microalgae were obtained, the microalgae were inoculated in a serum bottle containing 500 mL of BG-11 medium to form anmicroalga suspension with a microalga cell number of 1×10⁵. The formula of BG-11 medium is shown in Table 10 below.

The CaCl₂ concentration of the BG-11 medium used in the experimental group was 0.5 times, 1 times or 2 times that of the control group. The microalgae in the experimental group and the control group were cultured, and sampled at different culturing time points to count their cell numbers.

TABLE 9
Culture conditions of Haematococcus pluvialis

| Item | Condition | |
|---|---|---|
| Culture algae name | Haematococcus pluvialis | |
| Culture medium | BG-11 medium | |
| Culture temperature | 25° | C. |
| Illuminance for culturing | 20,000 | Lux |
| Culture volume | 500 | mL |

| | |
|---|---|
| Days for Culturing | 10 |

TABLE 10
BG-11 medium formula (control group)

| Drug name | Final concentration | |
|---|---|---|
| NaNO₃ | 1.76 | mM |
| K₂HPO₄ | 0.23 | mM |
| MgSO₄•H₂O | 0.3 | mM |
| CaCl₂•2H₂O | 0.24 | mM |
| Citric Acid•H₂O | 0.031 | mM |
| Ferric Ammonium Citrate | 0.021 | mM |
| Na₂EDTA•2H₂O | 0.0027 | mM |
| Na₂CO₃ | 0.19 | mM |

BG-11 trace metal solution (1,000X dilution, per 200 mL)

| | | |
|---|---|---|
| H₃BO₃ | 2.86 | g |
| MnC1₂•4H₂O | 1.81 | g |
| ZnSO₄•7H₂O | 0.222 | g |
| NaMoO_{4•}2H₂O | 0.39 | g |
| CuSO₄•5H₂O | 0.079 | g |
| Co(NO₃)₂6H₂O | 49.4 | mg |

A-5. Clustered Regularly Interspaced Short Palindromic Repeats/CRISPR-Associated Protein 9 (Cas9) Gene Editing

CRISPR/Cas9 gene editing technology is derived from the immune system found in bacteria, and through this technology, specific DNA sequences can be effectively identified and cut. This technology has a higher success rate and efficiency than other gene editing tools in the past, and this technology has been successfully applied to different species of organisms.

Ribonucleoprotein (RNP) type gene editing mechanism is that Cas9 protein with endonuclease function and a guide RNA (guide RNA, gRNA) designed for the target gene form into a ribonucleoprotein complex outside the cell and then the complex is sent into the cell to identify the target gene in the cell by the guide RNA, and the Cas9 protein performs DNA cutting on the target gene to achieve the editing function [Witt, M A, Corn, JE, & Carroll, D., 2017. Genome editing via delivery of Cas9 ribonucleoprotein. Methods, 121, 9-15.].

In the present disclosure, ribonucleoprotein-type CRISPR/Cas9 gene editing technology is used.

1. Design of Guide RNA

Based on the nucleotide sequence of target gene, two guide RNAs were designed for the target gene.

2. Electroporation for Chlamydomonas sp. and Subsequent Culture

(1) Concentrated TAP medium for Chlamydomonas sp. were prepared and mixed with the pre-cultured Chlamydomonas sp. obtained in the above section “A-2. Pre-culture of Chlamydomonas sp.” to make the medium reach the original concentration and the OD₆₈₀ value reach 0.1 and then the experimental culture could be performed.

(2) The suspension of Chlamydomonas sp. was cultured to a cell concentration of 1×10⁶-2×10⁶ cells/mL, and then centrifuged at 2,500 rpm for 5 minutes to remove the supernatant and obtain microalga cells.

(3) The microalga cells were re-suspended with GeneArt MAX Efficiency Transformation Reagent to obtain a microalga suspension with a final concentration of 2×10⁸-3×10⁸ cells/mL.

(4) 100 pmole guide RNA and 100 pmole Cas9 protein were mixed in a volume of 10 μL and reacted for 20 minutes to form a ribonucleoprotein complex solution, and then 250 μL of microalga suspension was added to the ribonucleoprotein complex solution mentioned above and allowed to stand at 2° C.-8° C. for 5 minutes to form a mixture solution.

(5) The electroporation parameters for the electroporation system Gene Pulser II were set to 500 V, 50 μF, 800Ω, and 260 μL of the mixture solution mentioned above was transferred to an electroporation cuvette that was previously cooled on ice to perform electroporation using the electroporation system to deliver the ribonucleoprotein complex mentioned above into cells (electroporation could be performed 1 to 2 times). After the electroporation was completed, the electroporation cuvette was allowed to stand for 15 minutes for cell recovery.

(5) The mixture solution in the electroporation cuvette was added to a 50 mL centrifuge tube containing TAP medium with 40 mM sucrose solution at room temperature, and incubated at 26° C. under illumination (5000 lux).

(6) After culturing for 14-16 hours, the centrifuge tube was centrifuged at 2,500 rpm for 5 minutes to collect microalga cells, and the microalga cells spread on an agar plate made of TAP medium Incubate at 26° C. for 5-7 days under illumination (5,000 lux) until obvious colonies appear.

A-6. High-Throughput Microalga Growth Analysis Platform Test

Pre-cultured Chlamydomonas algae were mixed with the TAP medium. When the algae concentration reaches an OD₆₈₀ value of about 0.1, the high-throughput microalga growth analysis platform with microplates can be used to detect growth condition of algae.

The pre-cultured alga suspension of gene-edited microalgae was inoculated into fresh TAP medium.

1.5 mL of the microalgae suspension for growth analysis mentioned above was injected into the 24-well plate (transparent), and the plate was cover by a cap, and the surroundings of the plate was fixed with transparent tape to culture the microalgae, in which aperture was remained to facilitate air in and out. The culture conditions are as follows. Culturing temperature: 25° C. constant temperature culture; stirring rate: 150 rpm; illumination: 24 hours per day (intensity 10,000 lux).

After culturing, the OD₆₈₀ value of each well was measured by EPOCH2 microplate spectrophotometer (BioTek) to evaluate the amount of microalgae in each well. A higher OD₆₈₀ value meant more microalgae content, and thus microalgae with a higher growth rate can be screened.

A-7. Analysis of Microalgae Growth and its Products (Growth Test in 500 mL Serum Bottle Culture)

1. Culture of Chlamydomonas sp.

(1) Microalga suspension cultured to a stationary phase was centrifuged at 5,000 rpm, and the obtained microalgae was inoculated into a 500 mL serum bottle containing medium according to a condition in which a microalga suspension was formed with an OD₆₈₀ value of about 1.

(2) The experimental group and the control group were cultured and sampled at different culture time points for cell count, volume measurement and product analysis. The culture conditions are as follows. Culturing volume: 500 mL (in serum bottle); culturing temperature: 25° C. constant temperature incubation; aerating gas: 1% CO₂ (1 vvm mixed air); stirring rate: 150 rpm; illumination: 24 hours per day (intensity 10,000 lux).

2. Cell Count and Cell Size Measurement

(1) About 10 μL of the microalga suspension sample was dropped on a glass slide and observed with a microscope at 100× magnification.

(2) 20 different fields of view were selected, and then Micro Counter 1300 microbial cell counter (Celeromics) was used to count the number of cells in each field of view and measure the size of cells in each field of view, and the average value is automatically calculated by the program.

3. Microalga Product Analysis-Chlorophyll Extraction and Measurement

3-1. Chlorophyll Extraction of Microalgae

(1) About 1 mL of microalga suspension sample was added to a 15 mL centrifuge tube and 10 mL of 90% acetone solution was added therein.

(2) After covering the centrifuge tube with aluminum foil to avoid light, the centrifuge tube was vortexed at high speed for 10 minute.

(3) The centrifuge tube was left to stand for several minutes, and then inverted several times.

(4) Next, the centrifuge tube was continued to vortex at high speed for 10 minutes, and then left to stand at 4° C. in dark for at least 2 hours, but not more than 24 hours, in which the centrifuge tube should be taken from 4° C. in dark and vortexed to mix the liquid therein at least once.

(5) After the standing mentioned above, the centrifuge tube was vortexed again to mix the liquid therein.

(6) After that, the centrifuge tube was centrifuged at 675×g for 15 minutes or at 1,000×g for 10 minutes.

(7) Finally, after the centrifuge tube was placed in dark and warmed to room temperature, the supernatant is taken, which was an extract containing chlorophyll.

3.2 Quantification by Spectrophotometer:

(1) Spectrophotometer was warmed up for more than 30 minutes. After that, the selected wavelengths (750 nm, 664 nm, 647 nm, and 630 nm) were set, and the instrument was zeroed with 90% acetone solution.

(2) After the extract was placed into the sample tank of the spectrophotometer, the absorbance values at the wavelengths of 750 nm, 664 nm, 647 nm and 630 nm were respectively measured and recorded.

(3) Concentration of chlorophyll A in the extract was calculated by the following equation.

Concentration of chlorophyll Ain an extract (Ca) (mg/L)=11.85 (OD₆₆₄−OD₇₅₀)−1.54 (OD₆₄₇−OD₇₅₀)−0.08 (OD₆₃₀−OD₇₅₀) Calculation equation:

OD₆₆₄ represents the absorbance at 664 nm; OD₇₅₀ represents the absorbance at 750 nm; OD₆₄₇ represents the absorbance at 647 nm; OD₆₃₀ represents the absorbance at 630 nm.

4. Microalgal product analysis-ultrasonic extraction (ultrasonic extraction), transesterification reaction and gas chromatography (GC) of microalgal oil and fat

4-1. Ultrasonic Extraction Method

0.1 g of sample was weighted and added to 6 mL of chloroform/methanol (chloroform:methanol=2:1) mixture solution, ultrasonicated for 1 hour, and then centrifuged at 4,500 rpm for 10 minutes. After the supernatant was taken out, the chloroform/methanol mixture solution was re-added to the remnant for repeated extraction until the sample appears colorless. After that, all the supernatant that was taken out were collected into a sample vial, sent to a fume hood and dried naturally to constant weight, and then weighted.

Lipid extraction rate %=(Weight of vial after oil extraction (g)−weight of empty vial (g))/Microalgal weight of sample (g)×100 Calculation formula of lipid extraction rate:

4-2. Transesterification of Microalgal Oil and Fat

(1) The crude fat extracted above was added to a 300 mL round bottom bottle, 20 mL of 0.5N KOH/MeOH solution was added therein, and reacted in an oil bath at 100° C. for 10 minutes.

(2) The round bottom bottle was removed from the oil bath to stop the solution from boiling.

(3) Next, 20 mL of 0.7 N HCl/MeOH solution and 10 mL of 14 wt % BF3/MeOH solution were added to the round bottom bottle mentioned above, and reacted at 100° C. for 10 minutes. After that, the round bottom bottle was left to stand to room temperature.

(4) 20 mL of n-heptane and 30 mL of saturated saline was added to the above round-bottomed bottle and mixed well, and ultrasonicated at room temperature for 5-10 minutes.

(5) After that, the round bottom bottle was centrifuged at 4,000 rpm for 15 minutes, and the liquid therein was poured into a 50 mL separating funnel, and the upper layer (fatty acid methyl ester (FAME)/n-heptane) was taken out.

4.3 Gas Chromatography Analysis

(1) 252.1 mg of standard methyl heptadecanoate was added to n-heptane to form a methyl heptadecanoate/n-heptane solution with a final concentration of 1 mg/mL. Then, 1 mL of the fatty acid methyl ester/n-heptane solution mentioned above and 0.1 mL of methyl heptadecanoate/n-heptane solution were thoroughly mixed by ultrasonication to form a mixture.

(2) 1.0 μL of the mixture mentioned above was injected into a gas chromatograph, and then the fatty acid methyl ester content was calculated through the integrated area, and the extraction rate thereof was compared with that of lipid obtained by ultrasonic extraction.

The calculation formula of fatty acid methyl ester content in gas chromatography analysis is as follows:

Fatty acid methyl ester wt %=[(Peak integral total area of C₁₄to C_{24:1}−area of C₁₇)/area of C₁₇]×C₁₇concentration(mg/mL)×C₁₇volume(mL)+Weigh of algal powder(mg)×Dilution ratio(20)×100

B. Experimental Results

1. Identification of Altered Target Genes for Microalga

1-1. Screening of Altered Target Genes in Haematococcus Pluvialis

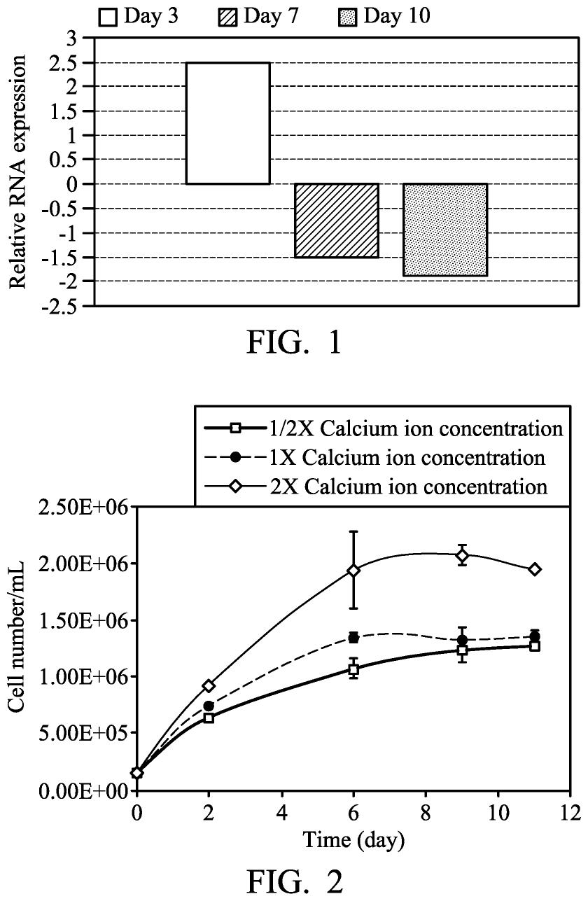

Haematococcus pluvialis was stimulated with the microalga growth promoter produced by the example of the Republic of China Patent No. 1630270, and then transcriptomics analysis was performed thereon to screen one of the genes of Haematococcus pluvialis which might be regulated by the microalga growth promoter, CAM1 gene. CAM1 gene encodes calmodulin 1, which is a multifunctional intermediate calcium binding messenger protein expressed in eukaryotic cells. The results are shown in FIG. 1.

1-2. The Effect of Calcium Ion Concentration of Culture Medium on the Growth of Haematococcus Pluvialis

To verify whether calcium ions affect cell growth or not, the calcium ion concentration in culture medium for Haematococcus pluvialis was adjusted, and changes in the cell number were detected.

The results are shown in FIG. 2.

According to FIG. 2, it is known that after increasing the calcium ion concentration of the culture medium by two times, the cell number of Haematococcus pluvialis increases by about 45%. This result confirms that the difference in calcium ion concentration will affect the growth of microalgae, and therefore it is concluded that by changing the performance of the CAM1 gene in microalgae, microalgae with improved growth rate and product production rate and/or yield should be obtained.

1-3. Confirmation of the Expression of CAM1 Gene in Chlamydomonas reinhardtii

In order to confirm whether the CAM1 gene in Chlamydomonas reinhardtii is also affected by the foregoing microalga growth promoter, the transcript database of Haematococcus pluvialis and the genome database Chlamydomonas sp. were cross-checked to confirm that Chlamydomonas reinhardtii has CAM1 gene.

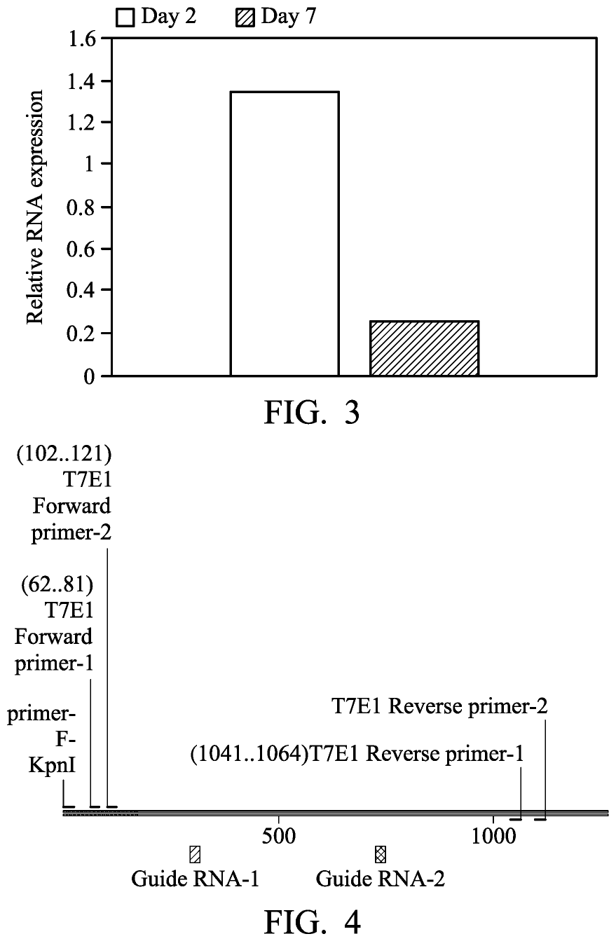

After that, Chlamydomonas reinhardtii was stimulated with the foregoing microalgae growth promoter, and then real-time quantitative polymerase chain reaction (RT-qPCR) was performed thereon to determine the expression level of CAM1 gene of Chlamydomonas reinhardtii. The results are shown in FIG. 3.

The results show that the expression level of the CAM1 gene of Chlamydomonas reinhardtii is also affected by the foregoing microalga growth promoter.

2. Clustered Regularly Interspaced Short Palindromic Repeats/CRISPR-Associated Protein 9 (Cas9) Gene Editing

First, the Genome FASTA sequence of the CAM1 gene of Chlamydomonas reinhardtii (SEQ ID NO. 1) was obtained from the database on the website of the National Center for Biotechnology Information (US)

Based on the foregoing sequence of the CAM1 gene of Chlamydomonas reinhardtii, two guide RNA sequences were designed, as shown in Table 11.

TABLE 11

| Item | Sequence | Exon position |
|---|---|---|
| Guide RNA 1 | GGTCAGCTGCTCGGTGTTCG | Exon 1 |
| | (SEQ ID NO. 8) | |
| Guide RNA 2 | CACGATTACCACCAAGGAGC | Exon 2 |
| | (SEQ ID NO. 9) | |

Compared to using a single guide RNA for gene editing alone, using two guide RNAs can greatly improve the efficiency of gene editing.

In addition, based on the CAM1 gene of Chlamydomonas reinhardtii, a polymerase chain reaction primer for screening the successfully edited microalgae strain in the subsequent T7 endonuclease I (T7E1) assay was designed. The primer sequences are shown in Table 12.

TABLE 12

| Item | Sequence |
|---|---|
| Forward | 5′-CAG AAC GGG GCG CCT TTG AG-3′ |
| primer | (SEQ ID NO. 10) |
| Reverse | 5′-CAT GCC GCA TGT CCA TGC AAG CTC-3′ |
| primer | (SEQ ID NO. 11) |

The position of the designed guide RNA sequence and the polymerase chain reaction primer used in the T7 endonuclease I (T7E1) assay relative to the CAM1 gene of Chlamydomonas reinhardtii is shown in FIG. 4.

The foregoing guide RNA and Cas9 protein were mixed to form a ribonucleoprotein (RNP) complex, and transfected into Chlamydomonas reinhardii cells by electroporation.

After electroporation, Chlamydomonas reinhardtii was smeared on a culture plate for culturing several days to wait for Chlamydomonas single colony to grow on the plate.

3. Screening of Chlamydomonas Gene Edited Strains by T7 Endonuclease I Assay

Colony PCR was performed one by one for each single colony to obtain the gene sequence containing the target fragment in each Chlamydomonas reinhardtii.

Cross-annealing was performed on the obtained fragments and gene fragment of the wild type. If the target gene undergoes gene editing to cause sequence variation, the gene sequence is different from the original gene sequence and cannot be completely matched, and T7 endonuclease I will cut the DNA fragment that cannot be completely matched to obtain the DNA fragments with specific sizes (Guschin, D Y, et. Al. (2010) A rapid and general assay for monitoring endogenous gene modification. Methods Mol Biol, 649, 247-256.). According to the position of the guide RNA designed by the present disclosure, it is expected that the target fragment may be cut into 2 to 3 fragment sizes of 216, 318, 414, 647 and 747 base pair lengths by T7 endonuclease I.

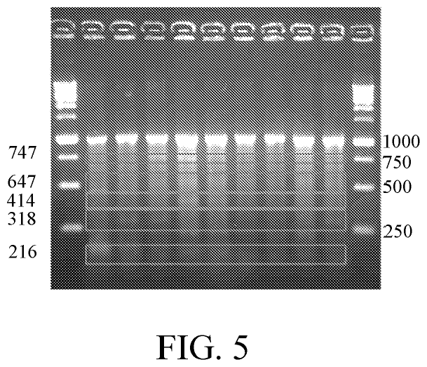

The obtained fragments and gene fragment of the wild type strain which were re-paired were cut with T7 endonuclease I, and then the size of the DNA fragments was analyzed by agar gel DNA electrophoresis (FIG. 5).

The colonies showing the expected DNA length were the candidate strains for subsequent growth analysis. 10 candidate strains which were possible to have been genetically edited were selected.

4. The Platform Test for High-Throughput Microalga Growth Analysis

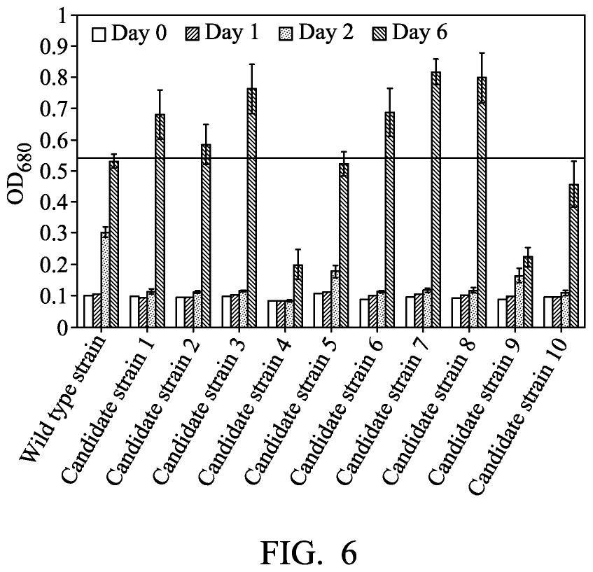

High-throughput microalga growth analysis for the foregoing candidate strains was performed in a 24-well plate. The preliminary analysis results show that the measured OD₆₈₀ values of candidate strains 1, 3, 6, 7 and 8 are significantly higher than that of the wild type strains (FIG. 6). Next, candidate strains 3, 7 and 8 with better growth performance were selected to perform growth test in 500 mL serum bottle culture.

5. Growth Test of 500 mL Serum Bottle Culture

Candidate strains 3, 7 and 8 were cultured in 500 mL serum bottles for 7 days. After that, the cell number, cell size, chlorophyll content and lipid content of these candidate strains 3, 7 and 8 were determined.

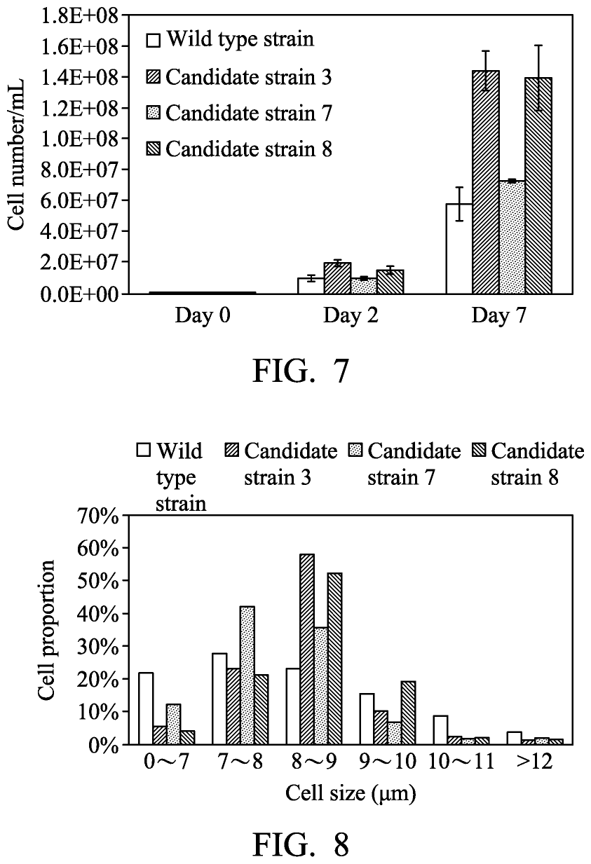

The results showed that the cell number of candidate strains 3 and 8 were significantly higher than that of the wild type strain, and were 150% and 140% higher than the wild type strain, respectively (FIG. 7).

In addition, in terms of changes in cell volume, it can be found that a population ratio for a cell size between 7-8 μm of candidate strains 3 and 8 with higher cell numbers was significantly higher than that of wild type strains (FIG. 8).

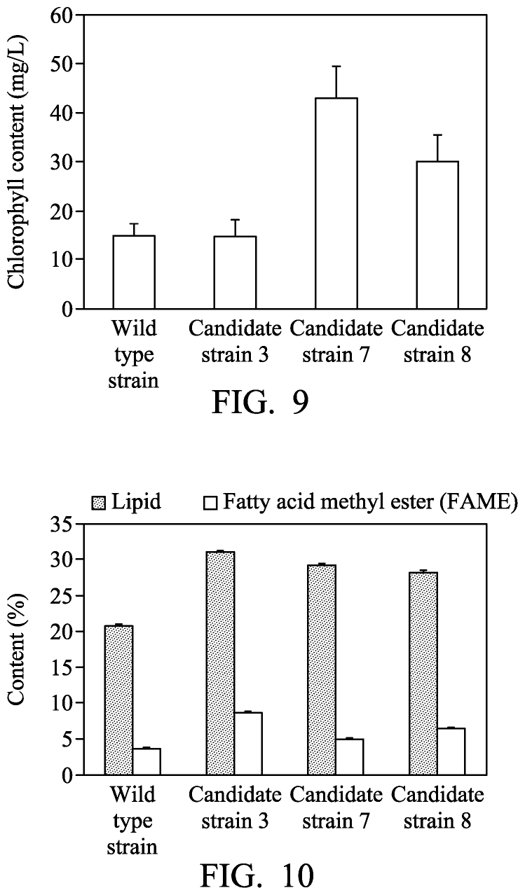

Furthermore, in terms of increase of chlorophyll content, candidate strain 7 had the best effect, which is 180% higher than that of wild type strain. Candidate strain 8 is 100% higher than wild type strain (FIG. 9).

In addition, in the lipid analysis, the lipid content of candidate strains 3, 7 and 8 were significantly higher than that of wild type strain, which were increased by 50%, 40% and 30%, respectively. Moreover, compared with wild type strains, the content of fatty acid methyl esters of candidate strains 3, 7 and 8 also increased by 138%, 38% and 77%, respectively (FIG. 10).

These results show that candidate strain 3 and candidate strain 8 both have an increased effect on growth and lipid content.

Candidate strain 3 and candidate strain 8 were named ITRI-ALG-3 and ITRI-ALG-8, respectively, and deposited at Bioresource Collection and Research Centre (BCRC) of Food Industry Research and Development Institute (FIRDI)(ROC.) on Dec. 27, 2019, and whose respective deposit numbers are BCRC 980055 and BCRC 980056.

6. Sequencing

Sequence changes on the gene editing target genes of candidate strains 3 and 8 were further analyzed.

The CAM1 genes of candidate strains 3 and 8 were sequenced. The sequence of CAM1 gene of candidate strain 3 is the sequence of SEQ ID NO. 4 (which encodes the amino acid sequence of SEQ ID NO. 6), and the sequence of CAM1 gene of candidate strain 8 is the sequence of SEQ ID NO. 5 (which encodes the amino acid sequence of the SEQ ID NO. 7).

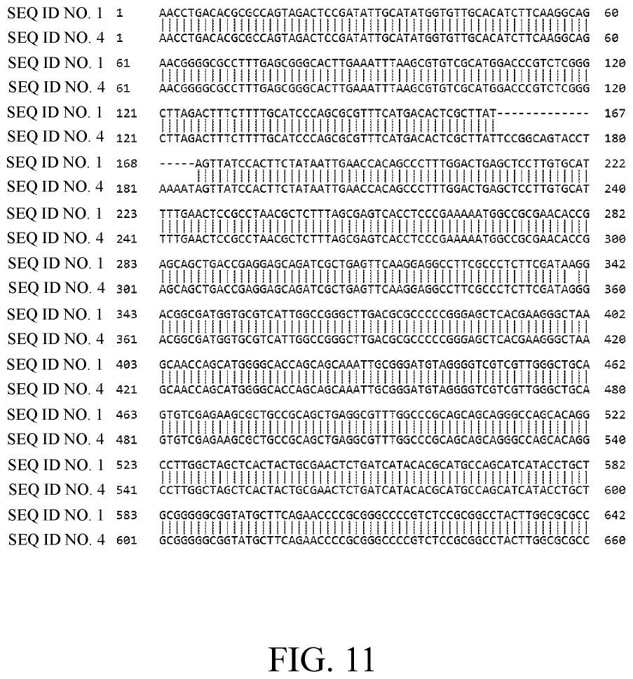

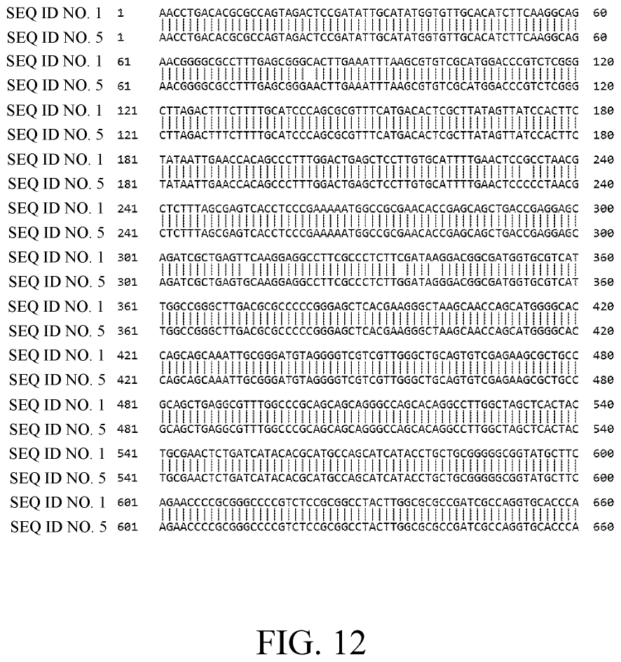

The sequence of the CAM1 gene of candidate strain 3 and the sequence of CAM1 gene of candidate strain 8 were respectively compared with the sequence of CAM1 gene of wild type strain, and the results are shown in FIG. 11 and FIG. 12.

According to FIG. 11, it is known that, relative to the sequence of the CAM1 gene of the wild type strain (SEQ ID NO. 1), candidate strain 3 has an insertion of a polynucleotide (the sequence of which is SEQ ID NO. 3) between position 167 and position 168, and the nucleotide at position 340 is substituted from A to G.

According to FIG. 12, it is known that, relative to the sequence of the CAM1 gene of the wild type strain (SEQ ID NO. 1), candidate strain 8 has a nucleotide substitution from C to A at position 82, a nucleotide substitution from G to C at position 233, a nucleotide substitution from T to G at position 313, a nucleotide substitution from C to G at position 335 and a nucleotide substitution from A to G at position 340.

Based on the results mentioned above, it is known that candidate strains 3 and 8 indeed have changed in the target region of gene editing, verifying that the superior performance of candidate strains 3 and 8 is indeed due to changes in the nucleotide and/or nucleotide sequence of the CAM1 gene.

It will be apparent to those skilled in the art that various modifications and variations can be made to the disclosed embodiments. It is intended that the specification and examples be considered as exemplary only, with the true scope of the disclosure being indicated by the following claims and their equivalents.

What is claimed is:

1. A method for increasing the yield of microalgae and the yield of a product produced by the microalgae, comprising:
performing a change procedure on CAM1 gene and/or calmodulin 1 encoded by the CAM1 gene in a microalga, such that a change occurs in a nucleotide and/or the nucleotide sequence of the CAM1 gene and/or an amino acid and/or the amino acid sequence of the calmodulin 1 encoded by the CAM1 gene in the microalga to obtain an altered microalga which has an altered CAM1 gene and/or an altered calmodulin 1,
wherein the altered microalga has a higher growth rate and a higher product production rate and/or yield than an unaltered microalga.

2. The method for increasing the yield of microalgae and the yield of a product produced by the microalgae as claimed in claim 1, wherein the change comprises substitution, insertion, frameshift, deletion and/or addition in a sequence, and/or comprises modification to a nucleotide and/or an amino acid.

3. The method for increasing the yield of microalgae and the yield of a product produced by the microalgae as claimed in claim 1, wherein the nucleotide sequence of the CAM1 gene comprises the sequence of SEQ ID NO. 1.

4. The method for increasing the yield of microalgae and the yield of a product produced by the microalgae as claimed in claim 1, wherein the amino acid sequence of the protein encoded by the CAM1 gene comprises the sequence of SEQ ID NO. 2.

5. The method for increasing the yield of microalgae and the yield of a product produced by the microalgae as claimed in claim 1, wherein the nucleotide sequence of the CAM1 gene comprises the sequence of SEQ ID NO. 1, and a region where the change occurs in the nucleotide sequence of the CAM1 gene comprises:
at least one of the regions of the sequence of SEQ ID NO. 1 in the following:
(i) a region between position 80 and position 90;
(ii) a region between position 160 and position 170;
(iii) a region between position 230 and position 240;
(iv) a region between position 310 and position 320;
(v) a region between position 330 and position 340; and
(vi) a region between position 335 and position 345.

6. The method for increasing the yield of microalgae and the yield of a product produced by the microalgae as claimed in claim 1, wherein the nucleotide sequence of the CAM1 gene comprises the sequence of SEQ ID NO. 1, and the change occurring in the nucleotide sequence of the CAM1 gene comprises:
at least one change occurring in the sequence of SEQ ID NO. 1 from among the following:
(i) a substitution of a nucleotide at position 82;
(ii) an insertion of at least one nucleotide between position 167 and position 168;
(iii) a substitution of a nucleotide at position 233;
(iv) a substitution of a nucleotide at position 313;
(v) a substitution of a nucleotide at position 335; and
(vi) a substitution of a nucleotide at position 340.

7. The method for increasing the yield of microalgae and the yield of a product produced by the microalgae as claimed in claim 6, wherein the substitution of a nucleotide at position 82 is a substitution from C to A, the insertion of at least one nucleotide between position 167 and position 168 is an insertion of a polynucleotide between position 167 and position 168, and the sequence of the polynucleotide is the sequence of SEQ ID NO. 3, the substitution of a nucleotide at position 233 is a substitution from G to C, the substitution of a nucleotide at position 313 is a substitution from T to G, the substitution of a nucleotide at position 335 is a substitution from C to G or the substitution of a nucleotide at position 340 is a substitution from A to G.

8. The method for increasing the yield of microalgae and the yield of a product produced by the microalgae as claimed in claim 1, wherein the nucleotide sequence of the CAM1 gene comprises the sequence of SEQ ID NO. 1, and the change occurring in the nucleotide sequence of the CAM1 gene comprises:
changes occurring in the sequence of SEQ ID NO. 1 in the following:
(i) an insertion of a polynucleotide between position 167 and position 168, wherein the sequence of the polynucleotide is the sequence of SEQ ID NO. 3; and
(ii) a substitution from A to G at position 340.

9. The method for increasing the yield of microalgae and the yield of a product produced by the microalgae as claimed in claim 1, wherein the nucleotide sequence of the CAM1 gene comprises the sequence of SEQ ID NO. 1, and the change occurring in the nucleotide sequence of the CAM1 gene comprises:
changes occurring in the sequence of SEQ ID NO. 1 among the following:
(i) a substitution from C to A at position 82;
(ii) a substitution from G to C at position 233;
(iii) a substitution from T to G at position 313;
(iv) a substitution from C to G at position 335; and
(v) a substitution from A to G at position 340.

10. The method for increasing the yield of microalgae and the yield of a product produced by the microalgae as claimed in claim 1, wherein the nucleotide sequence of the altered CAM1 gene comprises the sequence of SEQ ID NO. 4 or the sequence of SEQ ID NO. 5.

11. The method for increasing the yield of microalgae and the yield of a product produced by the microalgae as claimed in claim 1, wherein the amino acid sequence of the altered calmodulin 1 comprises the sequence of SEQ ID NO. 6 or the sequence of SEQ ID NO. 7.

12. The method for increasing the yield of microalgae and the yield of a product produced by the microalgae as claimed in claim 1, wherein the change procedure comprises clustered regularly interspaced short palindromic repeats/CRISPR-associated protein 9 gene editing, zinc-finger nucleases (ZFN) gene editing, or transcription activator-like effector nucleases (TALENs) gene editing.

13. The method for increasing the yield of microalgae and the yield of a product produced by the microalgae as claimed in claim 1, wherein a product produced by the microalgae comprises chlorophyll, a lipid, astaxanthin, a protein, a fatty acid, an amino acid, a carbohydrate, a vitamin, a compound, a cellulose, an enzyme, a colloid, diatomaceous earth, or a pigment.

14. The method for increasing the yield of microalgae and the yield of the microalgae as claimed in claim 1, wherein the microalgae comprises Chlamydomonas sp., Haematococcus sp., Synechococcus sp., Volvox sp., Emiliania huxleyi or Heterocapsa triquetra.

15. The method for increasing the yield of microalgae and the yield of the microalgae as claimed in claim 1, wherein the altered microalga is Chlamydomonas reinhardii ITRI-ALG-3 which is deposited at Bioresource Collection and Research Centre (BCRC) of Food Industry Research and Development Institute (FIRDI) and whose deposit number is BCRC 980055 or Chlamydomonas reinhardtii ITRI-ALG-8 which is deposited at Bioresource Collection and Research Centre of Food Industry Research and Development Institute and whose deposit number is BCRC 980056.

16. An altered CAM1 gene, of which the nucleotide sequence comprises the sequence of SEQ ID NO. 1, in which the nucleotide sequence of the CAM1 gene has a change, and a region having the change in the nucleotide sequence of the CAM1 gene comprises:
at least one of the regions of the sequence of SEQ ID NO. 1 in the following:
(i) a region between position 80 and position 90;
(ii) a region between position 160 and position 170;
(iii) a region between position 230 and position 240;
(iv) a region between position 310 and position 320;
(v) a region between position 330 and position 340; and
(vi) a region between position 335 and position 345.

17. The altered CAM1 gene as claimed in claim 16, wherein the change comprises:
at least one change of the nucleotide sequence of SEQ ID NO. 1 from among the following:
(i) a substitution of a nucleotide at position 82;
(ii) an insertion of at least one nucleotide between position 167 and position 168;
(iii) a substitution of a nucleotide at position 233;
(iv) a substitution of a nucleotide at position 313;
(v) a substitution of a nucleotide at position 335; and
(vi) a substitution of a nucleotide at position 340.

18. The altered CAM1 gene as claimed in claim 17, wherein the substitution of a nucleotide at position 82 is a substitution from C to A, the insertion of at least one nucleotide between position 167 and position 168 is an insertion of a polynucleotide between position 167 and position 168, and the sequence of the polynucleotide is the sequence of SEQ ID NO. 3, the substitution of a nucleotide at position 233 is a substitution from G to C, the substitution of a nucleotide at position 313 is a substitution from T to G, the substitution of a nucleotide at position 335 is a substitution from C to G or the substitution of a nucleotide at position 340 is a substitution from A to G.

19. The altered CAM1 gene as claimed in claim 16, wherein the change comprises:
changes of the sequence of SEQ ID NO. 1 in the following:
(i) an insertion of a polynucleotide between position 167 and position 168, wherein the sequence of the polynucleotide is the sequence of SEQ ID NO. 3; and
(ii) a substitution from A to G at position 340.

20. The altered CAM1 gene as claimed in claim 16, wherein the change comprises:
changes of the nucleotide sequence of SEQ ID NO. 1 in the following:
(i) a substitution from C to A at position 82;
(ii) a substitution from G to C at position 233;
(iii) a substitution from T to G at position 313;
(iv) a substitution from C to G at position 335; and
(v) a substitution from A to G at position 340.

21. The altered CAM1 gene as claimed in claim 16, wherein the nucleotide sequence of the altered CAM1 gene comprises the sequence of SEQ ID NO. 4 or the sequence of SEQ ID NO. 5.

22. The altered CAM1 gene as claimed in claim 16, wherein the altered CAM1 gene encodes a polypeptide, and the amino acid sequence of the polypeptide comprises the sequence of SEQ ID NO. 6 or the sequence of SEQ ID NO. 7.

23. A polypeptide, of which the amino acid sequence comprises the sequence of SEQ ID NO. 6 or the sequence of SEQ ID NO. 7.

24. A microalga, comprising an altered CAM1 gene and/or a polypeptide,
wherein the nucleotide sequence of the altered CAM1 gene comprises the sequence of SEQ ID NO. 1, in which the nucleotide sequence of the CAM1 gene has a change, and a region having the change in the nucleotide sequence of the CAM1 gene comprises:
at least one of the regions of the sequence of SEQ ID NO. 1 in the following:
(i) a region between position 80 and position 90;
(ii) a region between position 160 and position 170;
(iii) a region between position 230 and position 240;
(iv) a region between position 310 and position 320;
(v) a region between position 330 and position 340; and
(vi) a region between position 335 and position 345, and
wherein the amino acid sequence of the polypeptide comprises the sequence of SEQ ID NO. 6 or the sequence of SEQ ID NO. 7.

25. The microalga as claimed in claim 24, wherein the microalga comprises Chlamydomonas sp., Haematococcus sp., Synechococcus sp., Volvox sp., Emiliania huxleyi or Heterocapsa triquetra.

26. The microalga as claimed in claim 24, wherein the microalga is Chlamydomonas reinhardtih ITRI-ALG-3 which is deposited at Bioresource Collection and Research Centre (BCRC) of Food Industry Research and Development Institute (FIRDI) and whose deposit number is BCRC 980055 or Chlamydomonas reinhardtii ITRI-ALG-8 which is deposited at Bioresource Collection and Research Centre of Food Industry Research and Development Institute and whose deposit number is BCRC 980056.

27. A novel Chlamydomonas sp., which is Chlamydomonas reinhardtii ITRI-ALG-3 deposited at Bioresource Collection and Research Centre of Food Industry Research and Development Institute and whose deposit number is BCRC 980055 or which is Chlamydomonas reinhardtii ITRI-ALG-8 which is deposited at Bioresource Collection and Research Centre of Food Industry Research and Development Institute and whose deposit number is BCRC 980056.


SEQUENCE LISTING (11 sequences)

SEQ ID NO: 1 - DNA, 2092 bp, Chlamydomonas reinhardtii
aacctgacacgcgccagtagactccgatattgcatatggtgttgcacatcttcaaggcagaacggggcgcctttgagcgggcacttgaaatttaagcgtgtcgcatggacccgtctcgggcttagactttcttttgcatcccagcgcgtttcatgacactcgcttatagttatccacttctataattgaaccacagccctttggactgagctccttgtgcattttgaactccgcctaacgctctttagcgagtcacctcccgaaaaatggccgcgaacaccgagcagctgaccgaggagcagatcgctgagttcaaggaggccttcgccctcttcgataaggacggcgatggtgcgtcattggccgggcttgacgcgcccccgggagctcacgaagggctaagcaaccagcatggggcaccagcagcaaattgcgggatgtaggggtcgtcgttgggctgcagtgtcgagaagcgctgccgcagctgaggcgtttggcccgcagcagcagggccagcacaggccttggctagctcactactgcgaactctgatcatacacgcatgccagcatcatacctgctgcgggggcggtatgcttcagaaccccgcgggccccgtctccgcggcctacttggcgcgccgatcgccaggtgcacccagcgggcttgtaaccttcgctcgttgctctcctctgcttaacaggcacgattaccaccaaggagctggggacggtcatgcgctcgctgggccagaaccccaccgaggctgagctgcaagacatgatcagcgaggtggacgccgacggcaacggcaccattgatttccctgagttcctgatgctcatggcccgcaagatgaaggtgagcttccggagccggtcccacgcctgaaagtccaaaattgaccgtgtcctacctgcggtttacattttcaggagactgaccacgaggatgagctgcgggaagccttcaaggtgttcgataaggatggcaacggcttcatctcggccgctgaggtgagcttgcatggacatgcggcatgttgacggccgcgagcaggagggggcagcaattgcattcgtcgcctgtggggttcatgcggtggtttggagctgggctagagccatcagggaaacctagacgaagggtatgggtacgtgccgctttaagtcgtggaattcccactgacactccctgcgcattgaaccctcccgctgccagcttcggcacgtgatgaccaacctgggcgagaagctgtccgaggaggaggtcgatgagatgatccggtgggtgggatcaaaccaggctgggtgttggggtgctgctgcggaacacccgcgcagtgtcagggcagtgggagtagtttgtgcatgttggaggtgatagctgtggcgtccggctacgtctgacaactgcggttccgtttggtccgaacagggaggccgacgtggacggtgatggccaggtcaactacgaggtgagcttacttgggcgaaagaagaggggcttagacagcacgcccctatgtgtaacccgggacttctgttcctgctacgtgtttcaatccctggcaccatttaacgcgctggccgtcgtgcacttgacaggagttcgtgcgcatgatgacgtctggcgccactgacgacaaggacaagaagggccacaagtaaagcatttcactgtctgaagcagcacggacctgggcggaagttggacgccgtcgttgtaattgttggctggacggtgcgggcgttgccggatgagggggcatttggcccacgggctaacggggtgaagggttcagacgcaaatagcttcttggcccctgcaggaagcattttagacttagcggccagtagaaggctcgttgctccggacgaggcacagaggttcatggggatggatgcgggggcagcatgagactgatgacgcggttacatggggtggcttgccagccggactaattgaatcgcggtatatatggcacgtcggatagctgctgctttgcaaggtacagagtctatgactgcatacgatgtgctcatagcagcgccaggaacggcgctgcacacgtgtaactctttctgtacaa

SEQ ID NO: 2 - protein, 666 aa, Chlamydomonas reinhardtii
PDTRQTPILHMVLHIFKAERGAFERALEIACRMDPSRATFFCIPARFMTLAYSYPLLLNHSPLDAPCAFTPPNALRVTSRKMAANTEQLTEEQIAEFKEAFALFDKDGDGASLAGLDAPPGAHEGLSNQHGAPAANCGMGSSLGCSVEKRCRSGVWPAAAGPAQALASSLLRTLIIHACQHHTCCGGGMLQNPAGPVSAAYLARRSPGAPSGLVTFARCSPLLNRHDYHQGAGDGHALAGPEPHRGAARHDQRGGRRRQRHHFPVPDAHGPQDEGELPEPVPRLKVQNPCPTCGLHFQETDHEDELREAFKVFDKDGNGFISAAEVSLHGHAACRPRAGGGSNCIRRLWGSCGGLELGSHQGNLDEGYGYVPLVVEFPLTLPAHTLPLPASARDDQPGREAVRGGGRDDPVGGIKPGWVLGCCCGTPAQCQGSGSSLCMLEVIAVASGYVQLRFRLVRTGRPTWTVMARSTTRAYLGERRGATARPYVPGTSVPATCFNPWHHLTRWPSCTQEFVRMMTSGATDDKDKKGHKSISLSEAARTWAEVGRRRCNCWLDGAGVAGGGIWPTGRGEGFRRKLLGPCRKHFRLSGQKARCSGRGTEVHGDGCGGSMRLMTRLHGVACQPDLNRGIYGTSDSCCFARYRVYDCIRCAHSSARNGAAHVLFLY

SEQ ID NO: 3 - DNA, 18 bp, Artificial Sequence
  Insertion sequence
tccggcagtacctaacct

SEQ ID NO: 4 - DNA, 2230 bp, Artificial Sequence
  Altered CAM1 gene
aacctgacacgcgccagtagactccgatattgcatatggtgttgcacatcttcaaggcagaacggggcgcctttgagcgggcacttgaaatttaagcgtgtcgcatggacccgtctcgggcttagactttcttttgcatcccagcgcgtttcatgacactcgcttattccggcagtacctaacctagttatccacttctataattgaaccacagccctttggactgagctccttgtgcattttgaactccgcctaacgctctttagcgagtcacctcccgaaaaatggccgcgaacaccgagcagctgaccgaggagcagatcgctgagttcaaggaggccttcgccctcttcgatagggacggcgatggtgcgtcattggccgggcttgacgcgcccccgggagctcacgaagggctaagcaaccagcatggggcaccagcagcaaattgcgggatgtaggggtcgtcgttgggctgcagtgtcgagaagcgctgccgcagctgaggcgtttggcccgcagcagcagggccagcacaggccttggctagctcactactgcgaactctgatcatacacgcatgccagcatcatacctgctgcgggggcggtatgcttcagaaccccgcgggccccgtctccgcggcctacttggcgcgccgatcgccaggtgcacccagcgggcttgtaaccttcgctcgttgctctcctctgcttaacaggcacgattaccaccaaggagctggggacggtcatgcgctcgctgggccagaaccccaccgaggctgagctgcaagacatgatcagcgaggtggacgccgacggcaacggcaccattgatttccctgagttcctgatgctcatggcccgcaagatgaaggtgagcttccggagccggtcccacgcctgaaagtccaaaattgaccgtgtcctacctgcggtttacattttcaggagactgaccacgaggatgagctgcgggaagccttcaaggtgttcgataaggatggcaacggcttcatctcggccgctgaggtgagcttgcatggacatgcggcatgttgaccgccgcgagcaggagggggcagcaattgcattcgtcgcctgtggggttcatgcggtggtttggagctgggctagagccatcagggaaacctagacgaagggtatgggtacgatgccgctttaagtcgtggaattcccactgacactccctgcgcattgaaccctcccgctgccagcttcggcacgtgatgaccaacctgggcgagaagctgtccgaggaggaggtcgatgagatgatccggtgggtgggatcaaaccaggctgggtgttggggtgctgctgcggaacacccgcgagtgtcagggcagtgggagtagtttgtgcatgttggaggtgatagctgtggcgtccggctacgtctgacaactgcggttccgtttggtccgaacagggaggccgacgtggacggtgatggccaggtcaactacgaggtgagcttacttgggcgaaagaagaggggcttagacagcacgcccctatgtgtaacccgggacttctgttcctgctacgtgtttcaatccctggcaccatttaacgcgctggccgtcgtgcacttgacaggagttcgtgcgcatgatgacgtctggcgccactgacgacaaggacaagaagggccacaagtaaagcatttcactgtctgaagcagcacggacctgggcggaagttggacgccgtcgttgtaattgttggctggacggtgcgggcgttgccggatgagggggcatttggcccacgggctaacggggtgaagggttcagacgcaaatagcttcttggcccctgcaggaagcattttagacttagcggccagtagaaggctcgttgctccggacgaggcacagaggttcatggggatggatgcgggggcagcatgagactgatgacgcggttacatggggtggcttgccagccggactaattgaatcgcggtatatatggcacgtcggatagctgctgctttgcaaggtacagagtctatgactgcatacgatgtgctcatagcagcgccaggaacggcgctgcacacgtgtaactctttctgtacaaggcgcctcaaagaccgcaatgtgcacgaaacgtgccgaaccaggtcacgctgcaagtggccaatgccagcaagctgtgaactccagccttcaagcagttttgccagtgccagctctgcag

SEQ ID NO: 5 - DNA, 2212 bp, Artificial Sequence
  Altered CAM1 gene
aacctgacacgcgccagtagactccgatattgcatatggtgttgcacatcttcaaggcagaacggggcgcctttgagcgggaacttgaaatttaagcgtgtcgcatggacccgtctcgggcttagactttcttttgcatcccagcgcgtttcatgacactcgcttatagttatccacttctataattgaaccacagccctttggactgagctccttgtgcattttgaactccccctaacgctctttagcgagtcacctcccgaaaaatggccgcgaacaccgagcagctgaccgaggagcagatcgctgagtgcaaggaggccttcgccctcttggatagggacggcgatggtgcgtcattggccgggcttgacgcgcccccgggagctcacgaagggctaagcaaccagcatggggcaccagcagcaaattgcgggatgtaggggtcgtcgttgggctgcagtgtcgagaagcgctgccgcagctgaggcgtttggcccgcagcagcagggccagcacaggccttggctagctcactactgcgaactctgatcatacacgcatgccagcatcatacctgctgcgggggcggtatgcttcagaaccccgcgggccccgtctccgcggcctacttggcgcgccgatcgccaggtgcacccagcgggcttgtaaccttcgctcgttgctctcctctgcttaacaggcacgattaccaccaaggagctggggacggtcatgcgctcgctgggccagaaccccaccgaggctgagctgcaagacatgatcagcgaggtggacgccgacggcaacggcaccattgatttccctgagttcctgatgctcatggcccgcaagatgaaggtgagcttccggagccggtcccacgcctgaaagtccaaaattgaccgtgtcctacctgcggtttacattttcaggagactgaccacgaggatgagctgcgggaagccttcaaggtgttcgataaggatggcaacggcttcatctcggccgctgaggtgagcttgcatggacatgcggcatgttgaccgccgcgagcaggagggggcagcaattgcattcgtcgcctgtggggttcatgcggtggtttggagctgggctagagccatcagggaaacctagacgaagggtatgggtacgatgccgctttaagtcgtggaattcccactgacactccctgcgcattgaaccctcccgctgccagcttcggcacgtgatgaccaacctgggcgagaagctgtccgaggaggaggtcgatgagatgatccggtgggtgggatcaaaccaggctgggtgttggggtgctgctgcggaacacccgcgagtgtcagggcagtgggagtagtttgtgcatgttggaggtgatagctgtggcgtccggctacgtctgacaactgcggttccgtttggtccgaacagggaggccgacgtggacggtgatggccaggtcaactacgaggtgagcttacttgggcgaaagaagaggggcttagacagcacgcccctatgtgtaacccgggacttctgttcctgctacgtgtttcaatccctggcaccatttaacgcgctggccgtcgtgcacttgacaggagttcgtgcgcatgatgacgtctggcgccactgacgacaaggacaagaagggccacaagtaaagcatttcactgtctgaagcagcacggacctgggcggaagttggacgccgtcgttgtaattgttggctggacggtgcgggcgttgccggatgagggggcatttggcccacgggctaacggggtgaagggttcagacgcaaatagcttcttggcccctgcaggaagcattttagacttagcggccagtagaaggctcgttgctccggacgaggcacagaggttcatggggatggatgcgggggcagcatgagactgatgacgcggttacatggggtggcttgccagccggactaattgaatcgcggtatatatggcacgtcggatagctgctgctttgcaaggtacagagtctatgactgcatacgatgtgctcatagcagcgccaggaacggcgctgcacacgtgtaactctttctgtacaaggcgcctcaaagaccgcaatgtgcacgaaacgtgccgaaccaggtcacgctgcaagtggccaatgccagcaagctgtgaactccagccttcaagcagttttgccagtgccagctctgcag

SEQ ID NO: 6 - protein, 710 aa, Artificial Sequence
  Protein encoded by altered CAM1 gene
PDTRQTPILHMVLHIFKAERGAFERALEIACRMDPSRATFFCIPARFMTLAYSGSTPSYPLLLNHSPLDAPCAFTPPNALRVTSRKMAANTEQLTEEQIAEFKEAFALFDRDGDGASLAGLDAPPGAHEGLSNQHGAPAANCGMGSSLGCSVEKRCRSGVWPAAAGPAQALASSLLRTLIIHACQHHTCCGGGMLQNPAGPVSAAYLARRSPGAPSGLVTFARCSPLLNRHDYHQGAGDGHALAGPEPHRGAARHDQRGGRRRQRHHFPVPDAHGPQDEGELPEPVPRLKVQNPCPTCGLHFQETDHEDELREAFKVFDKDGNGFISAAEVSLHGHAACPPRAGGGSNCIRRLWGSCGGLELGSHQGNLDEGYGYDAALSRGIPTDTPCALNPPAASFGTPTWARSCPRRRSMRSGGWDQTRLGVGVLLRNTRECQGSGSSLCMLEVIAVASGYVQLRFRLVRTGRPTWTVMARSTTRAYLGERRGATARPYVPGTSVPATCFNPWHHLTRWPSCTQEFVRMMTSGATDDKDKKGHKSISLSEAARTWAEVGRRRCNCWLDGAGVAGGGIWPTGRGEGFRRKLLGPCRKHFRLSGQKARCSGRGTEVHGDGCGGSMRLMTRLHGVACQPDLNRGIYGTSDSCCFARYRVYDCIRCAHSSARNGAAHVLFLYKAPQRPQCARNVPNQVTLQVANASKLTPAFKQFCQCQLC

SEQ ID NO: 7 - protein, 705 aa, Artificial Sequence
  Protein encoded by altered CAM1 gene
PDTRQTPILHMVLHIFKAERGAFERELEIACRMDPSRATFFCIPARFMTLAYSYPLLLNHSPLDAPCAFTPPNALRVTSRKMAANTEQLTEEQIAECKEAFALLDRDGDGASLAGLDAPPGAHEGLSNQHGAPAANCGMGSSLGCSVEKRCRSGVWPAAAGPAQALASSLLRTLIIHACQHHTCCGGGMLQNPAGPVSAAYLARRSPGAPSGLVTFARCSPLLNRHDYHQGAGDGHALAGPEPHRGAARHDQRGGRRRQRHHFPVPDAHGPQDEGELPEPVPRLKVQNPCPTCGLHFQETDHEDELREAFKVFDKDGNGFISAAEVSLHGHAACPPRAGGGSNCIRRLWGSCGGLELGSHQGNLDEGYGYDAALSRGIPTDTPCALNPPAASFGTPTWARSCPRRRSMRSGGWDQTRLGVGVLLRNTRECQGSGSSLCMLEVIAVASGYVQLRFRLVRTGRPTWTVMARSTTRAYLGERRGATARPYVPGTSVPATCFNPWHHLTRWPSCTQEFVRMMTSGATDDKDKKGHKSISLSEAARTWAEVGRRRCNCWLDGAGVAGGGIWPTGRGEGFRRKLLGPCRKHFRLSGQKARCSGRGTEVHGDGCGGSMRLMTRLHGVACQPDLNRGIYGTSDSCCFARYRVYDCIRCAHSSARNGAAHVLFLYKAPQRPQCARNVPNQVTLQVANASKLTPAFKQFCQCQLC

SEQ ID NO: 8 - DNA, 20 bp, Artificial Sequence
  Guide RNA
ggtcagctgctcggtgttcg

SEQ ID NO: 9 - DNA, 20 bp, Artificial Sequence
  Guide RNA
cacgattaccaccaaggagc

SEQ ID NO: 10 - DNA, 20 bp, Artificial Sequence
  Forward primer
cagaacggggcgcctttgag

SEQ ID NO: 11 - DNA, 24 bp, Artificial Sequence
  Reverse primer
catgccgcatgtccatgcaagctc